(19) United States
(12) Patent Application Publication
(10) Pub. No.: US 2005/0106195 A1
(43) Pub. Date: May 19, 2005

(54) N-octanoyl amino acid composition for slimming the human body

(76) Inventor: Christine Garcia, Castres (FR)

Correspondence Address: Linda K. Russell;Air Liquide, Suite 1800, 2700 Post Oak Blvd., Houston, TX, 77056, US

(21) Appl. No.: 10/783,001

(22) Filed: Feb. 20, 2004

(30) Foreign Application Priority Data
Feb. 21, 2003 (FR) 0302162

(51) Int. Cl. A61K 38/08; A61K 38/06; A61K 38/04; A61K 38/05; A61K 7/00

(52) U.S. Cl. 424/401; 514/17; 514/18; 514/19

(57) ABSTRACT

Use of a compound of formula (I): 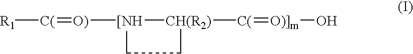
in which R₁ represents a hydrocarbon radical comprising 7 carbon atoms, R₂ represents the characterizing chain of an amino acid and m is between 1 and 50, or of a mixture of the said compounds of formula (I), as a slimming active agent. Nontherapeutic method of treatment using the said compound and use of the said compound for preparing a medicament with lipolytic activity, intended to induce slimming of the human body.

The subject of the present invention is a novel use of cosmetic active agents for slimming the human body.

Some of the fat in the human body is stored in the form of triglycerides, in cells of the fatty tissue of the dermis, called adipocytes. Slimming reflects a reduction in the fat stored in the adipocytes. This process requires a preliminary step which takes place inside these cells and which consists in hydrolysing the triglycerides to fatty acids and glycerol. This phenomenon is called lipolysis.

Most slimming cosmetic formulations currently marketed contain at least one compound possessing a lipolytic activity. The one most frequently used is caffeine, but theophylline is also known to possess such a property.

During their search for novel active agents with lipolytic activity which have better compatibility with the skin than those of the state of the art, the inventors demonstrated that certain N-acylated derivatives of amino acids known for their soothing property also had a lipolytic property which was more effective than that of caffeine.

Accordingly, according to a first aspect, the subject of the invention is the use of a compound of formula (I): 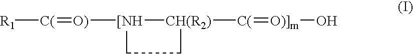
in which R₁ represents a linear or branched, saturated or unsaturated, aliphatic hydrocarbon radical comprising 7 carbon atoms, R₂ represents the characterizing chain of an amino acid and m is between 1 and 50, or of a mixture of the said compounds of formula (I), as a slimming active agent, in a composition containing a cosmetically acceptable medium.

The compound of formula (I) as defined above may be in the form of a free acid or in a partially or completely salified form. When the compound of formula (I) is in a salified form, it comprises in particular alkali metal salts such as the sodium, potassium or lithium salts, alkaline-earth metal salts such as the calcium, magnesium or strontium salts; an ammonium salt or a salt of an amino alcohol such as the (2-hydroxyethyl)-ammonium salt. It may also comprise metal salts such as divalent zinc or manganese salts, trivalent iron, lanthanum, cerium or aluminium salts. In general, the degree of salification of the compound of formula (I) as defined above will additionally depend on its pK_{A} and the salt concentration of the composition into which it is incorporated.

In the following disclosure, the expression compound of formula (I) is understood to mean a compound of formula (I) in free form or in a partially or completely salified form.

The expression “characterizing chain” used to define the radical R₂ denotes the nonfunctional principal chain of the amino acid considered.

Thus, for an amino acid represented by general formula (IIIa):
H₂N—CH(R₂)—C(═O)—OH (IIIa)
and for a cyclic amino acid represented by formula (IIIb): 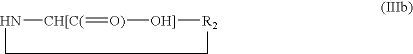
the characterizing chain will be the chain represented by R₂.

R₂ represents in particular the characterizing chain of an amino acid chosen from glycine, alanine, serine, aspartic acid, glutamic acid, valine, threonine, arginine, lysine, proline, leucine, phenylalanine, isoleucine, histidine, tyrosine, tryptophan, asparagine, glutamine, cysteine, cystine, methionine, hydroxyproline, hydroxylysine, sarcosine or ornithine.

The subject of the invention is mainly the use of a compound of formula (I) as defined above, in which, in the residue:
—HN—CH(R₂)—C(═O)— (IIIa)
R₂ represents the characterizing chain of glycine.

The subject of the invention is more particularly the use of a compound of formula (I) as defined above, in which m is a decimal number between 1 and 10 and it is preferably less than 5.

According to a most particular aspect of the present invention, in formula (I) as defined above, m is less than or equal to 2 and is more particularly less than or equal to 1.4.

According to another most particular aspect of the present invention, in formula (I) as defined above, m is equal to 1.

According to another particular variant of the present invention, a single compound of formula (I) as defined above is used in the composition containing the cosmetically acceptable medium.

According to another particular variant of the present invention, a mixture of compounds of formula (I) as defined above is used.

The compounds of formulae (I) are generally obtained by N-acylation of compounds of formulae (IIIa) or (IIIb), as defined above, or their salts.

In the case of a mixture of compounds of formulae (I) it is for example obtained by N-acylation of the mixture of amino acids resulting from the total or partial hydrolysis of proteins of any origin.

These proteins may be of animal origin, such as for example collagen, elastin, fish flesh protein, fish gelatin, keratin or casein, of plant origin, such as cereal, flower or fruit proteins such as for example the proteins derived from soya bean, sunflower, oats, wheat, maize, barley, potato, lupin, field bean, sweet almond, kiwi, mango or apple; they may also be proteins obtained from Chorella (unicellular algae), pink algae, yeasts or silk.

This hydrolysis is carried out, for example, by heating to temperatures of between 60 and 130° C. a protein placed in an acidic or alkaline medium.

This hydrolysis may also be carried out enzymatically, with a protease, optionally coupled with a post-alkaline or post-acid hydrolysis. When m is greater than 1, R₂ represents a single chain or several chains characterizing different amino acids, depending on the protein hydrolysed and the degree of hydrolysis.

The aminograms of a few proteins of plant origin are presented in the following tables:

TABLE 1

| | Origin of the protein | | | |
|---|---|---|---|---|
| | (proportions of amino acids expressed | | | |
| | in wt %) | | | |
| | Oats | Soya bean | Wheat | Sunflower |
| Glycine | 6.9 | 4.2 | 3.2 | 6.2 |
| Alanine | 5.9 | 4.2 | 2.6 | 4.8 |
| Serine | 5.6 | 5.1 | 1.7 | 5.1 |
| Aspartic acid | 16.2 | 11.7 | 3.4 | 10.6 |
| Glutamic acid | 28.3 | 19.1 | 37.9 | 23.6 |
| Valine | 2.9 | 5.0 | 4.2 | 4.8 |
| Threonine | 3.1 | 3.9 | 2.7 | 4.4 |
| Arginine | 6.6 | 7.8 | 3.7 | 8.4 |
| Lysine | 3.6 | 6.2 | 1.9 | 3.2 |
| Proline | 4.7 | 5.4 | 11.7 | 3.0 |
| Leucine | 6.4 | 8.1 | 7.1 | 6.4 |
| Phenylalanine | 1.4 | 5.0 | 5.4 | 4.3 |
| Isoleucine | 2.2 | 4.8 | 3.7 | 4.1 |
| Histidine | 1.7 | 2.6 | 2.4 | 2.0 |
| Tyrosine | 1.5 | 3.5 | 3.1 | 2.7 |
| Methionine | 1.2 | 1.2 | 1.6 | 1.8 |
| Cysteine/cystine | 1.9 | 1.5 | 1.9 | 1.9 |
| Tryptophan | — | 1.0 | 1.0 | 1.3 |

TABLE 2

| | Origin of the protein | | | |
|---|---|---|---|---|
| | (proportions of amino acids expressed | | | |
| | in wt %) | | | |
| | Lupin | Potato | Field bean | Maize |
| Glycine | 0.9 | 4.8 | 4.0 | 2.4 |
| Alanine | 2.4 | 5.0 | 4.0 | 7.95 |
| Serine | 6.1 | 5.8 | 4.9 | 5.1 |
| Aspartic acid | 15.8 | 12.5 | 10.5 | 10.6 |
| Glutamic acid | 8.0 | 11.5 | 16.8 | 23.6 |
| Valine | 7.9 | 7.1 | 4.5 | 4.8 |
| Threonine | 8.1 | 6.1 | 3.6 | 4.4 |
| Arginine | 16.1 | 5.0 | 9.21 | 8.4 |
| Lysine | 7.1 | 7.8 | 6.5 | 6.2 |
| Proline | — | 5.1 | 4.4 | 3.0 |
| Leucine | 7.45 | 10.4 | 7.4 | 8.1 |
| Phenylalanine | 8.6 | 6.4 | 4.4 | 4.3 |
| Isoleucine | 8.7 | 6.1 | 3.9 | 4.1 |
| Histidine | — | 2.2 | 2.6 | 2.0 |
| Tyrosine | — | 5.7 | 3.6 | 2.7 |
| Methionine | 0.6 | 2.4 | 0.8 | 1.8 |
| Cysteine/cystine | — | 1.6 | 1.7 | 1.9 |
| Tryptophan | 1.2 | 1.4 | 1.2 | 1.3 |
| Ornithine | 0.4 | — | — | — |

The acylation reaction is known to persons skilled in the art. It is described for example in international application published under the number WO 98/09611. It is carried out indifferently on an amino acid or on a mixture of amino acids. The acylating agent generally consists of an activated derivative of a carboxylic acid of formula R₁—C(═O)—OH, in which R₁ is as defined above, such as a symmetric anhydride of this acid, the methyl ester of this acid, or an acid halide such as the acid chloride or the acid bromide.

The subject of the invention is also a nontherapeutic method of treating the human body intended for slimming it, characterized in that a composition containing a cosmetically acceptable medium and an effective quantity of at least one compound of formula (I) as defined above, is applied to it.

The subject of the invention is also the use of at least one compound of formula (I), as defined above, for preparing a medicament with lipolytic activity, intended for inducing slimming of the human body.

In the compositions defined above, the compound of formula (I) is generally used in a quantity of between 0.01% and 10% of their weight, more particularly between 0.1% and 5% of their weight, and most particularly between 1% and 5% of their weight.

As the examples show, the compounds used in the cosmetic or therapeutic treatments defined above are characterized, unexpectedly, by a lipolytic activity greater than the compositions of the state of the art. They are therefore in general appropriate for the slimming treatments of the human body.

The compositions used in the said treatments are generally provided in the form of dilute aqueous or aqueous-alcoholic solutions, in the form of simple or multiple emulsions, such as water-in-oil (W/O), oil-in-water (O/W) or water-in-oil-in-water (W/O/W) emulsions in which the oil is of a vegetable or mineral nature, or in powdered form. They may also be dispersed or impregnated onto textile or onto nonwoven materials such as wipes, paper serviettes or clothing.

The compositions used in the said treatments are administered to the subject in the conventional forms used in cosmetics and in pharmacy; this includes more particularly topical, oral or parenteral administrations.

In general, the compounds of formula (I) are combined with many types of adjuvants or active ingredients used in cosmetic formulations, such as fatty substances, organic solvents, thickeners, gelling agents, emollients, antioxidants, opacifiers, stabilizers, foaming agents, perfumes, emulsifiers, which are ionic or nonionic, fillers, sequestrants, chelators, preservatives, chemical screening agents or inorganic screening agents, essential oils, colouring matter, pigments, hydrophilic or lipophilic active agents, humectants, for example glycerin, preservatives, colorants, perfumes, cosmetic active agents, inorganic or organic sunscreens, inorganic fillers such as iron oxides, titanium oxides and talc, synthetic fillers such as nylons and poly(methyl methacrylate) which are crosslinked or otherwise, silicone elastomers, sericites or plant extracts or alternatively lipid vesicles or any other ingredient customarily used in cosmetics.

As examples of oils which may be combined with the compound of formula (I), there may be mentioned mineral oils such as paraffin oil, liquid paraffin, isoparaffins or white mineral oils, oils of animal origin, such as squalene or squalane, vegetable oils, such as sweet almond oil, copra oil, castor oil, jojoba oil, olive oil, rapeseed oil, groundnut oil, sunflower oil, wheat germ oil, maize germ oil, soya bean oil, cottonseed oil, lucerne oil, poppy seed oil, pumpkinseed oil, evening primrose oil, millet oil, barley oil, rye oil, safflower oil, candlenut oil, passionflower oil, hazelnut oil, palm oil, shea butter, apricot kernel oil, calophyllum oil, sysymbrium oil, avocado oil, calendula oil; ethoxylated vegetable oils; synthetic oils such as fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, monoglycerides, diglycerides and triglycerides of fatty acids such as glyceryl triheptanoate, alkyl benzoates, poly-alpha-olefins, polyolefins such as polyisobutene, synthetic isoalkanes such as isohexadecane, isododecane, per-fluorinated oils and silicone oils. Among the latter, there may be mentioned more particularly dimethylpoly-siloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluorinated groups, cyclic silicones and silicones modified with alkyl groups.

As other fatty substances which may be combined with this active agent, there may be mentioned fatty alcohols or fatty acids.

Among the thickening and/or emulsifying polymers used in the present invention are for example homopolymers or copolymers of acrylic acid or of acrylic acid derivatives, homopolymers or copolymers of acrylamide, homopolymers or copolymers of acrylamide derivatives, homopolymers or copolymers of acrylamidomethylpropane-sulphonic acid, vinyl monomer, trimethylaminoethyl-acrylate chloride, hydrocolloids of plant or biosynthetic origin, for example xanthan gum, karaya gum, carrageenans, alginates; silicates; cellulose and its derivatives; starch and its hydrophilic derivatives; polyurethanes. Among the polymers of the polyelectrolyte type which may be used in the production of a gelled aqueous phase capable of being used in the preparation of W/O emulsions containing the compounds of formula (I) which are the subject of the present invention, there are for example copolymers of acrylic acid and 2-methyl-[(1-oxo-2-propenyl)amino]-1-propanesulphonic acid (AMPS), copolymers of acrylamide and 2-methyl-[(1-oxo-2-propenyl)amino]-1-propane-sulphonic acid, copolymers of 2-methyl-[(1-oxo-2-propenyl)amino]-1-propanesulphonic acid and (2-hydroxyethyl) acrylate, homopolymer of 2-methyl-[(1-oxo-2-propenyl)amino]-1-propanesulphonic acid, homopolymer of acrylic acid, copolymers of acryloyl-ethyltrimethylammonium chloride and acrylamide, copolymers of AMPS and vinylpyrrolidone, copolymers of acrylic acid and alkyl acrylates whose carbon chain comprises between ten and thirty carbon atoms, copolymers of AMPS and alkyl acrylates whose carbon chain comprises between ten and thirty carbon atoms. Such polymers are marketed respectively under the names SIMULGEL™ EG, SEPIGEL™ 305, SIMULGEL™ NS, SIMULGEL™ 800 and SIMULGEL™ A by the applicant.

Among the waxes which can be used in the present invention, there may be mentioned for example beeswax; carnauba wax, candelilla wax; ouricoury wax; Japan wax; cork fibre or sugarcane wax; paraffin waxes; lignite waxes; microcrystalline waxes; lanolin wax; ozokerite; polyethylene wax; hydrogenated oils; silicone waxes; vegetable waxes; fatty alcohols and fatty acids which are solid at room temperature; glycerides which are solid at room temperature. Among the emulsifiers which can be used in the present invention, there may be mentioned for example fatty acids, ethoxylated fatty acids, fatty acid esters of sorbitol, ethoxylated fatty acid esters, polysorbates, polyglycerol esters, ethoxylated fatty alcohols, sucrose esters, alkyl polyglycosides, sulphated and phosphated fatty alcohols or mixtures of alkyl polyglycosides and fatty alcohols described in French Patent Applications 2 668 080, 2 734 496, 2 756 195, 2 762 317, 2 784 680, 2 784 904, 2 791 565, 2 790 977, 2 807 435 and 2 804 432.

As examples of an active ingredient which may be combined with the compound of formula (I) there may be mentioned compounds having a lightening or depigmenting action, such as for example arbutin, kojic acid, hydroquinone, ellagic acid, vitamin C, magnesium ascorbyl phosphate, extracts of polyphenols, grape extracts, pine extracts, wine extracts, olive extracts, marc extracts, N-acylated proteins, N-acylated peptides, N-acylated amino acids, partial hydrolysates of N-acylated proteins, amino acids, peptides, total hydrolysates of proteins, partial hydrolysates of proteins, polyols (for example glycerin or butylene glycol), urea, pyrrolidonecarboxylic acid or derivatives of this acid, glycyrrhetinic acid, alpha-bisabolol, sugars or sugar derivatives, polysaccharides or their derivatives, hydroxy acids, for example lactic acid, vitamins, vitamin derivatives such as retinol, vitamin E and its derivatives, minerals, enzymes, coenzymes such as Coenzyme Q10, hormones or hormone-like substances, soya bean extracts, for example Raffermine™, wheat extracts, for example Tensine™ or Gliadine™, vegetable extracts such as extracts rich in tannins, extracts rich in isoflavones or extracts rich in terpenes, extracts of fresh water or marine algae, essential waxes, bacterial extracts, minerals, lipids in general, lipids such as ceramides or phospholipids, active agents having a slimming action such as caffeine or its derivatives, active agents having an antimicrobial activity or a purifying action in relation to greasy skins such as LIPACIDE™ PVB, active agents having an energizing or stimulating property such as SEPITONIC™ M3 or Physiogenyl™, panthenol and its derivatives such as SEPICAP™ MP, antiageing active agents such as SEPILIFT™ DPHP, LIPACIDE™ PVB, SEPIVINOL™, SEPIVITAL™, moisturizing active agents such as SEPICALM™ S, SEPICALM™ VG and LIPACIDE™ DPHP, “antiphotoageing” antiageing active agents, active agents protecting the integrity of the dermoepidermal junction, active agents increasing the synthesis of the components of the extracellular matrix, active agents having a slimming, toning or draining activity such as caffeine, theophylline, cAMP, green tea, sage, ginko biloba, ivy, horse-chestnut, bamboo, ruscus, butcher's broom, centella asiatica, heather, meadowsweet, fucus, rosemary, willow, active agents creating a sensation of “heat” on the skin, such as activators of skin microcirculation (for example nicotinates) or products creating a sensation of “freshness” on the skin (for example menthol and its derivatives).

As sunscreen which may be incorporated into the composition according to the invention, there may be mentioned all those which appear in the amended cosmetics directive 76/768/EEC, annex VII.

The following experimental study illustrates the invention without, however, limiting it.

A) Evaluation in Vitro of the Lipolytic Activity of the Compounds of Formula (I)

(1)—Aim and Principle of the Method

The objective of the experiment is to demonstrate, in an in vitro model of isolated human adipocytes, the lipolytic activity of the compounds used. This is because triglycerides are stored in the adipocytes and constitute the fat reserve. For this reserve to diminish, which is the desired aim when slimming products are used, the triglycerides must be hydrolysed so that the fatty acids can be removed from the cell. Hydrolysis of the triglycerides to nonesterified fatty acids and glycerol is called lipolysis.

The method described consists in incubating the products in the presence of human adipocytes in suspension, followed by the measurement of the nonesterified fatty acid level in the adipocyte incubation medium. The free fatty acid level thus reflects the hydrolysis of the triglycerides present in the adipocytes.

(2)—Experimental Protocol

(i) Cellular Model:

The test is carried out using human adipocytes isolated and prepared as a cellular suspension. The adipocytes are isolated from the subcutaneous abdominal adipose tissue recovered during plastic surgery operations (abdominal plastic surgery operations) performed on women. The cells are isolated from fresh tissue. The adipose tissue is isolated and dissociated by the action of a collagenase (SIGMA™, 1 mg/ml, 30 minutes at 37° C., gentle stirring). Collagenase digests the connective tissue present in the adipose tissue. After digestion, the cells are filtered and washed in an appropriate culture medium containing MEM medium free of phenol red, free of glutamine (SIGMA)+2.2 mg/ml of sodium bicarbonate (GIBCO)+50 IU of penicillin (BIOWHITTAKER™)+50 μg/ml of streptomycin (BIOWHITTAKER™)+1% (v/v) of L-glutamine (BIOWHITTAKER™)+0.5% of lipid-free serum albumin (SIGMA™). The adipocyte suspension is used immediately after its preparation.

(ii) Incubation of the Products with the Adipocytes

The test products are diluted in the adipocyte culture medium. They are incubated with the cells in suspension for two hours at 37° C. (250 μl of product+250 μl of adipocyte suspension).

(3)—Evaluation of the Results

After the incubation, the cell lysis is checked visually by the presence of a lipid layer at the surface of the cellular suspension. The supernatant media are collected. The free fatty acids are assayed by spectrophotometry using a commercial kit (NEFA™ C kit, WAKO), with reference to a fatty acid calibration series. The lypolytic activity of the products is evaluated relative to a control group incubated in the presence of adipocytes and in the absence of product. The reactivity of the adipocytes is systematically checked for the measurement of the lipolytic activity of the reference products, caffeine (1,3,7-trimethyl-xanthine) and theophylline (1,3-dimethyl-2,6-dihydroxy-purine). Five assays are performed for each of the test products. The lipolytic activity of LIPACIDE™ C8G, which contains active material 100% N-octanoylglycine, was thus evaluated.

The results of the trials, expressed by the arithmetic means of the five assays carried out for each of the products, are presented in the following table:

| | | | Lipolytic |
|---|---|---|---|
| | | Free fatty | activity |
| | Incubation | acid | (compared |
| | concentration | concentration | with the |
| | (wt %/volume) | (μM) | control = 100) |
| Control | — | 11.54 ± 2.71 | 100 |
| Caffeine | 0.0019 | 15.10 ± 4.11 | 131 |
| Theophylline | 0.0019 | 16.53 ± 4.23 | 143 |
| LIPACIDE ™ C8G | 0.0001 | 23.83 ± 5.11 | 206 |
| LIPACIDE ™ C8G | 0.0005 | 25.30 ± 2.70 | 219 |
| LIPACIDE ™ C8G | 0.0010 | 25.75 ± 0.86 | 223 |

These results show that while the slimming compositions of the state of the art (caffeine and theophylline) act on lipolysis with a multiplication factor of 1.3 to 1.4 relative to the control, that according to the invention, that is to say comprising at least one compound of formula (I) as defined above, acts with a factor of 2.06 to 2.23 at lower concentrations.

B)—Examples of Cosmetic Formulations

In the following examples, the proportions are expressed as percentages by weight.

### EXAMPLE 1

### Slimming Body Milk

| | |
|---|---|
| MONTANOV ™ L | 3.00% |
| Phytosqualane | 8.00% |
| Sweet almond oil | 2.00% |
| Water | qsp 100% |
| SEPIGEL ™ 501 | 1.50% |
| LIPACIDE ™ C8G | 3.00% |
| SEPICIDE ™ CI | 0.20% |
| SEPICIDE ™ HB | 0.30% |
| Perfume | 0.30% |

### EXAMPLE 2

### Anti-Sagging Cream (Oval Target of the Face)

| | |
|---|---|
| MONTANOV ™ 202 | 3.50% |
| MONTANOV ™ 14 | 1.00% |
| SEPILIFT ™ DPHP | 1.00% |
| LANOL ™ 1688 | 15.00% |
| Wheat germ oil | 5.00% |
| Water | qsp 100% |
| SIMULGEL ™ EG | 1.30% |
| LIPACIDE ™ C8G | 2.00% |
| SEPICIDE ™ CI | 0.20% |
| SEPICIDE ™ HB | 0.30% |
| Perfume | 0.10% |

### EXAMPLE 3

### Anti-Plumpness Spray

| | |
|---|---|
| MONTANE ™ 60 | 3.30% |
| MONTANOX ™ 60 | 1.70% |
| Caprylic/capric triglycerides | 6.00% |
| Isohexadecane | 5.00% |
| Magnesium Aluminium Silicate | 1.50% |
| Water | qsp 100% |
| SIMULGEL ™ EG | 1.00% |
| LIPACIDE ™ C8G | 2.00% |
| Centalla asiatica/hydrocotyle | 1.00% |
| extract | |
| SEPICIDE ™ CI | 0.20% |
| SEPICIDE ™ HB | 0.30% |
| Perfume | 0.40% |
| Water | qsp 100% |

### EXAMPLE 4

### Refreshing Slimming Gel

| | |
|---|---|
| SEPIGEL ™ 305 | 3.50% |
| Hydroxyethylcellulose | 1.00% |
| Caffeine | 5.00% |
| Menthol | 0.30% |
| Ethanol | 50.00% |
| LIPACIDE ™ C8G | 3.00% |
| SEPICIDE ™ LD | 1.00% |
| Perfume | 0.20% |
| Water | qsp 100% |

### EXAMPLE 5

### Slimming Body Fluid

| | |
|---|---|
| SIMULGEL ™ NS | 2.50% |
| Xanthan gum | 0.20% |
| LANOL ™ 99 | 5.00% |
| LIPACIDE ™ C8G | 2.00% |
| Ginkgo biloba extract | 2.00% |
| Cola extract | 1.00% |
| Ginseng extract | 0.50% |
| SEPICIDE ™ HB | 1.50% |
| Perfume | 0.10% |
| Water | qsp 100% |

### EXAMPLE 6

### Toning Revitalizing Lotion Intended to be Impregnated into Body Wipes

| | |
|---|---|
| LIPACIDE ™ C8G | 1.50% |
| Glycerin | 5.00% |
| Ethanol | 5.00% |
| Ruscus extract | 3.00% |
| SEPITONIC ™ M3 | 1.00% |
| SEPICIDE ™ CI | 0.20% |
| SEPICIDE ™ HB | 0.30% |
| Water | qsp 100% |

### EXAMPLE 7

### Slimming Shower Gel

| | |
|---|---|
| MONTALINE ™ C40 | 8.00% |
| PROTEOL ™ OAT | 5.00% |
| Sodium lauryl sulphate | 9.00% |
| LIPACIDE ™ C8G | 3.00% |
| Green tea extract | 1.00% |
| KATHON ™ CG | 0.80 |
| Green colorant | qs |
| Green tea perfume | 1.00% |
| Lactic acid | qs pH = 6.5 |
| Water | qsp 100% |

### EXAMPLE 8

### Biphasic Disinfiltrating Massage

| | |
|---|---|
| Arabic coffee oil | 1.00% |
| LANOL ™ 189 | 20.00% |
| LANOL ™ 99 | 10.00% |
| Borage oil | 2.00% |
| Perfume | 0.10% |
| LIPACIDE ™ C8G | 3.00% |
| Glycerin | 3.00% |
| Ethanol | 10.00% |
| Blue colorant | qs |
| Water | qsp 100% |

The definitions of the commercial products used in the examples are the following:
SEPILIFT™ DPHP: (INCI name: Dipalmitoyl hydroxyproline), marketed by the company SEPPIC;
SEPICIDE™ CI: Imidazoline urea (preservative), marketed by the company SEPPIC;
SEPICIDE™ HB: Mixture of phenoxyethanol, methylparaben, ethylparaben, propylparaben and butylparaben (preservative), marketed by the company SEPPIC;
SEPICIDE™ LD: Phenoxyethanol marketed by the company SEPPIC;
KATHON™ CG: (INCI name: methyl isothiazolinone/Methyl chloroisothiazolinone);
MONTANE™ 60: Sorbitan stearate;
MONTANOX™ 60: Polysorbate 60;
SIMULGEL™ EG: Self-reversible invert latex of copolymer such as those described in international publication WO 99/36445 (INCI name: Sodium acrylate/Sodium acryloyldimethyl taurate copolymer and Isohexadecane and Polysorbate 80) marketed by the company SEPPIC;
SIMULGEL™ NS: Self-reversible invert latex of copolymer such as those described in international publication WO 99/36445 (INCI name: hydroxyethylacrylate/Sodium acryloyldimethyl taurate copolymer and squalane and Polysorbate 60) marketed by the company SEPPIC;
SEPIGEL™ 305: Self-reversible invert latex (INCI name: Polyacrylamide/C13-14 Isoparaffin/Laureth-7);
SEPIGEL™ 501: Self-reversible invert latex INCI name: C13-14 Isoparaffin/Mineral Oil/Sodium polyacrylate-/Polyacrylamide/Polysorbate 85;
LANOL™ 99 Isononyl isononanoate marketed by the company SEPPIC;
LANOL™ 189: Isostearyl isononanoate
LANOL™ 1688: Cetearyl ethyl hexanoate marketed by the company SEPPIC;
SEPITONIC™ M3: Mixture of magnesium aspartate, copper gluconate and zinc gluconate marketed by the company SEPPIC;
MONTALINE™ C40: Cocamidopropyl betainamide MEA chloride
PROTEOL™ OAT: Sodium Lauroyl oat amino acids;
MONTANOV™ 14: Myristyl alcohol/Myristyl glucoside;
MONTANOV™ L: Emulsifying agent based on a C14-C22 alcohol and a C12-C20 alkyl polyglucoside such as those described in European Patent Application EP 0 995 487;
MONTANOV™ 202 is an emulsifying agent based on arachidyl alcohol, behenyl alcohol and arachidyl polyglucoside.

1-8. (canceled)

9. A method of utilizing a composition as a slimming agent in a formulation containing a cosmetically acceptable medium, wherein said composition is represented by formula (I): 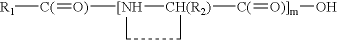
wherein R₁ comprises at least one linear or branched, saturated or unsaturated, aliphatic hydrocarbon radical comprising 7 carbon atoms,
wherein R₂ comprises an amino acid chain, and
wherein m is in the range of from about 1 to about 50.

10. The method according to claim 9, wherein said formula (I) is in at least one form selected from the group consisting of:
a) free acid,
b) partially salified, and
c) completely salified.

11. The method according to claim 10, wherein said salified formula (I) is produced using at least one salt selected from the group consisting of:
a) alkali metal salts,
b) alkaline-earth metal salts,
c) ammonium salts,
d) salts of amino alcohols,
e) divalent metal salts, and
f) trivalent metal salts.

12. The method according to claim 9, wherein the amino acid of said amino acid chain is represented by formula (IIIa):
H2N—CH(R2)—C(═O)—H

13. The method according to claim 9, wherein the amino acid of said amino acid chain is represented by formula (IIIb):

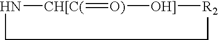

14. The method according to claim 9, wherein said R₂ comprises at least one component selected from the group consisting of:
a) glycine,
b) alanine,
c) serine,
d) aspartic acid,
e) glutamic acid,
f) valine,
g) threonine,
h) arginine,
i) lysine,
j) proline,
k) leucine,
l) phenylalanine,
m) isoleucine,
n) histidine,
o) tyrosine,
p) tryptophan,
q) asparagine,
r) glutamine,
s) cysteine,
t) cystine,
u) methionine,
v) hydroxyproline,
w) hydroxylysine,
x) sarcosine, and
y) ornithine.

15. The method according to claim 14, wherein said R₂ is a glycine chain.

16. The method according to claim 15, wherein said glycine chain is represented by formula (IIIa):
—HN—CH(R₂)—C(═O)—

17. The method according to claim 9, wherein said m is in the range of from about 1 to about 10.

18. The method according to claim 17, wherein said m is less than about 5.

19. The method according to claim 18, wherein said m is less than or equal to about 2.

20. The method according to claim 19, wherein said m is less than or equal to about 1.4.

21. The method according to claim 20, wherein said m is equal to about 1.

22. The method according to claim 9, wherein said method is administered by at least one method selected from the group consisting of:
a) topically,
b) orally, and
c) parenterally.

23. The method according to claim 22, wherein said composition is administered in the range of from about 0.01% to about 10% by weight.

24. The method according to claim 23, wherein said range is from about 0. 1% to about 5%.

25. The method according to claim 24, wherein said range is from about 1% to about 5%.

26. The method according to claim 23, wherein said composition is in at least one form selected from the group consisting of:
a) dilute aqueous,
b) aqueous-alcoholic,
c) simple emulsion, and
d) multiple emulsion.

27. The method according to claim 9, wherein said composition may be dispersed or impregnated onto textile or nonwoven materials.

28. The method according to claim 9, wherein said composition is added to at least one formulation selected from the group consisting of:
a) fatty substances,
b) organic solvents,
c) thickeners,
d) gelling agents,
e) emollients,
f) antioxidants,
g) opacifiers,
h) stabilizers,
i) foaming agents,
j) perfumes,
k) emulsifiers,
l) fillers,
m) sequestrants,
n) chelators,
o) preservatives.
p) chemical screening agents,
q) inorganic screening agents,
r) essential oils,
s) colouring matter,
t) pigments,
u) hydrophilic,
v) lipophilic active agents, and
w) humectants.

29. A method for preparing a formulation intended for slimming the human body comprising the step of:
i) introducing into a cosmetically acceptable medium, a composition represented by formula (I): 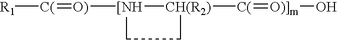
wherein R₁ comprises at least one linear or branched, saturated or unsaturated, aliphatic hydrocarbon radical comprising 7 carbon atoms,
wherein R₂ comprises an amino acid chain, and
wherein m is in the range of from about 1 to about 50; and
ii) producing said formulation.

30. The method according to claim 29, wherein said composition formula (I) is obtained by conducting a partial or total hydrolysis of a protein.

31. The method according to claim 30, wherein said protein is selected from the group consisting of:
a) collagen,
b) elastin,
c) fish flesh protein,
d) fish gelatin,
e) keratin,
f) casein,
g) cereal,
h) flower,
i) fruit proteins,
j) soya bean,
k) sunflower,
l) oats,
m) wheat,
n) maize,
o) barley,
p) potato,
q) lupin,
r) field bean,
s) sweet almond,
t) kiwi,
u) mango,
v) apple;
w) Chorella (unicellular algae),
x) pink algae,
y) yeasts, and
z) silk.

32. The method according to claim 30, wherein said hydrolysis occurs at an operating temperature in the range of from about 60° to about 130° C.

33. The method according to claim 30, wherein said hydrolysis is carried out enzymatically with a protease.

34. The method according to claim 30, wherein said hydrolysis is coupled with a post-alkaline or a post-acid.
